(19) United States
(12) Patent Application Publication
(10) Pub. No.: US 2023/0338081 A1
(43) Pub. Date: Oct. 26, 2023

(54) SYSTEM AND METHOD FOR MINIMALLY INVASIVE TREATMENT WITH INJECTABLE ELECTRODES

(71) Applicant: NEURONOFF, INC., Cleveland, OH (US)

(72) Inventors: Stephan NIEUWOUDT, Cleveland, OH (US); Manfred FRANKE, Cleveland, OH (US); David BOLUS, Cleveland, OH (US); Derrick LIU, Cleveland, OH (US); Emily SZABO, Cleveland, OH (US); Shaher AHMAD, Cleveland, OH (US); Aniruddha UPADHYE, Cleveland, OH (US); Sean ZUCKERMANN, Lakewood, OH (US); Craig WATSON, Cleveland Heights, OH (US); Andre SNELLINGS, Cleveland, OH (US); Amelia HOWE, Cleveland, OH (US)

(21) Appl. No.: 18/026,705

(22) PCT Filed: May 19, 2021
(86) PCT No.: PCT/US2021/033265

§ 371 Date: Mar. 16, 2023

Related U.S. Application Data
(60) Provisional application No. 63/079,275, filed on Sep. 16, 2020.
(60) Provisional application No. 63/119,444, filed on Nov. 30, 2020.
(60) Provisional application No. 63/153,223, filed on Feb. 24, 2021.
(60) Provisional application No. 63/167,836, filed on Mar. 30, 2021.
(60) Provisional application No. 63/171,780, filed on Apr. 7, 2021.
(60) Provisional application No. 63/184,656, filed on May 5, 2021.

(51) Int. Cl. A61B 18/14; A61B 18/16

(52) U.S. Cl. CPC A61B 18/1482; A61B 18/148; A61B 18/16; A61B 2018/144; A61B 2018/0072

(57) ABSTRACT

The invention is a system and method of minimally invasive treatment with wire structure electrodes dispensed without open cut downs or laparoscopy and using energy forms including radiofrequency, microwave, direct current, and high intensity focused ultrasound.

### STATEMENT REGARDING RELATED APPLICATIONS

This application claims priority to, and the full benefit of, U.S. provisional application No. 63/079,275 filed on Sep. 16, 2020; international application #PCT/US20/061374 filed Nov. 19, 2020; U.S. provisional application 63/119,444 filed Nov. 30, 2020; U.S. provisional application No. 63/153,223 filed Feb. 24, 2021; U.S. provisional application No. 63/167,836 filed Mar. 30, 2021; U.S. provisional application No. 63/171,780 filed on Apr. 7, 2021, U.S. provisional application No. 63/184,656 filed on May 5, 2021 and international application PCT/US21/33007 filed on May 18, 2021. This application also incorporates in their entirety both international application #PCT/US20/061374 filed on Nov. 19, 2020 (referred to herein as PCT '374) and international application PCT/US21/33007, filed on May 18, 2021 (referred to herein as PCT '007), as if both were set forth herein.

### FIELD OF THE INVENTION

The field of the invention is minimally invasive treatment of tissue with injectable electrodes comprising wire structures.

### ASPECTS OF THE INVENTION

The present invention includes methods of treating a tissue target with an array of wire structure electrodes including those which are non-helical (rolled, folded, extruded, twisted, braided as in PCT '374) which are very mechanically compliant when injected against or into bodily tissue, or using a helical wire structure electrode (PCT '007) that is less mechanically compliant but able to form a bunching anchor 8 in a deterministic fashion when injected against, around, onto, or into biological tissue. Examples of the non-helical structure are shown in FIGS. 4-C, 8-10, and 29-30, and examples of the helical structure are in FIGS. 11-15, 18-A, 20, 22-28

The wire structure electrodes of PCT '374 and PCT '007 have certain similarities. They all are made of fine wire. All are injectable through a dispenser (e.g., a needle) in a minimally invasive procedure without an open cut down or even laparoscopy. They can be injected in a linear fashion when the needle is being retracted to form a linear path (or curved out of a curved needle). All can, to some extent, bend, flex and fold and integrate well with the tissue and offer large surface area to provide ample interface for energy exchange.

Differences between helical and non-helical wire structure electrodes can be used by the clinician for different applications in ablation and other energy transfer therapies. Non-helical does not bend as deterministically around a corner and creates a deterministic filling in that it will fill a cavity but it will not necessarily widen the cavity in a way that helical will. Non-helical is more compressible than helical. Wire to air (compaction) inside the needle (prior to deployment) is around 60 to 70% for helical whereas the wire to air ratio inside the needle for non-helical is about 20 to 30%. Helical is mechanically stronger against deformation from outside forces and will tend to bend over instead of compressing during normal body movements (not so during removal). Non-helical is therefor a good choice near delicate structures that a clinician may not want to compress. Helical may compress a delicate structure if too much helical is injected into a cavity, thereby providing more pressure on its surrounding walls of said cavity. Non-helical is more easily conformable than helical. Helical can be easily injected from a thin cannula and it will form a more deterministic shape with rolling and folding over to form bunching anchors 8. Non-helical is less deterministic in its folding. Helical will not compress when ejected from a needle against mechanical pressure (say, when the dispenser is stationary) and form a bunching anchor, but non-helical will compress when ejected from a needle. Helical can help to widen a cavity during the injection, but non-helical will instead fold in on itself, making it ideal around delicate structures. Non-helical can be made to compress more in certain locations during the placement to concentrate wire there for either increasing charge injection or easing the interfacing by having a more densely packed non-helical in certain locations. Helical can be unzipped coil-by-coil for easy removal but non-helical does not unzip and is less easily removed in a chronic stage once that tissue has grown into it. Both though are easily removed if still in linear shape on injection day (meaning non-helical may compress but can still be pulled out before tissue in-growth, so can helical via unzipping even if it has formed an anchor. Helical can self-anchor during the placement procedure. This does not necessarily increase the wire density in that location, but instead will increase the cavity volume and thus target area for needle interfacing or volume of cavity that electrical energy may be deployed from into the tissue for stimulation or ablation applications.

### BRIEF DESCRIPTION OF THE FIGURES

Note: This application incorporates two PCT applications and adopts the reference numbers from PCT/US21/33007 in the following figures filed in this application.

FIG. 1-A is an image of a prior art electroporation probe and FIG. 1-B is the companion generator, Nanoknife, by AngioDynamics.

FIG. 2 contains four images of prior art devices. FIG. 2-A is an image of a 3 cm Single Active-Tip (Covidien Cool-Tip). FIG. 2-B is a StarBurst Expandable Electrode (Angiodynamics). FIG. 2-C is a Cluster Tri-Electrode 2.5 cm Active Tip (Covidien Cool-Tip). FIG. 2-D is a LeVeen Expandable Anchor Electrode (Boston Scientific).

FIG. 3-A is an image of cadaver tissue with the end of a prior art RF ablation probe inserted to a subcutaneous point in the tissue to be ablated. FIG. 3-B is an image of the same cadaver after ablation and removal of the probe and showing the pattern of ablation is limited to near the end of the probe.

FIG. 4-A is an image of cadaver tissue with a wire structure electrode implanted subcutaneously prior to RF ablation. FIG. 4-B shows the end of a gold wire in the wire structure electrode excised and clamped directly to an RF probe. FIG. 4-C is an image of the ablation pattern in the cadaver tissue from the wire structure electrode.

FIG. 5 is a photo showing a comparison of ablation patterns from the prior art device in FIG. 3-B and from a wire structure electrode in FIG. 4-C for the same duration and same amount of RF energy.

FIG. 6 is an ultrasound visualization of a pattern of RF ablation with the present invention for 20 watts and 120 seconds.

FIG. 7-A is transdermal imaging of a subcutaneously implanted gold wire structure electrode in a J-hook shape, and FIG. 7-B is the same image with cross sections 8-8, 9-9 and 10-10 labeled.

FIG. 8 is an ultrasound image of the cross section V-V in FIG. 7-B showing none of the wire structure electrode, taken with a VEVO 3100 system and 45 MHz probe.

FIG. 9 is an ultrasound image of the cross section W-W in FIG. 7-B showing only the long shaft of the J, taken with a VEVO 3100 system and 45 MHz probe.

FIG. 10 is an ultrasound image of the cross section X-X in FIG. 7-B showing the long shaft and hook of the J, taken with a VEVO 3100 system and 45 MHz probe.

FIG. 11 is an image (15.6×) of one embodiment of the injectable electrode with the helical wire structure after removal of the guidewire.

FIG. 12 is a closer image (100×) of a middle portion of the helical wire structure of FIG. 11.

FIG. 13 is a closer image (100×) of a rounded end of the helical wire structure of FIG. 11.

FIG. 14 is an image (300×) of a latitudinal cross-section of an electrode comprising a helical wire structure comprising a wire rope comprising 100 strands of 25 micron diameter gold wire.

FIG. 15 is the same electrode as in FIG. 14 before the 0.25 mm guidewire was removed.

FIG. 16 is an RF ablation experimental set-up.

FIG. 17 is a DC ablation experimental set-up.

FIG. 18 addresses post-experiment image-based temperature estimation in application of RF energy. FIG. 18-A is an image of an ablative zone effected by a 2 cm-length helical wire structure electrode subjected to 40 W power over minutes. FIG. 18-B shows temperature estimation based on RGB value found 2 mm from electrode site compared to RGB value of control set across 25-70 degrees Celsius. FIG. 18-C shows temperature estimation vs. distance at sites 1-5 mm from the center of the ablative zone.

FIG. 19 shows prior electrode placement and heat dispersion—bird's eye view. FIG. 19-A shows linear electrode placement, radial heat dispersion for a small (<3 cm) tumor, and complete ablation.

FIG. 20 is an image of rat liver left lobe subject to 1 cm helical wire structure electrode placement and RFA at 20 W over 1 minute, and the ablation pattern.

FIGS. 21-A, 21-B and 21-C are three schematics showing the benefit of “around a corner” procedures for a tumor (in dotted lines) shielded by a blood vessel comparing prior art ablation devices and outcomes with the present invention and outcome, compared to the prior art.

FIG. 22 contains four images of patterns of radiofrequency ablation for linear helical wire structures in tissue-mimicking polyacrylamide gel phantoms laced with a thermochromic ink, along with a temperature scale.

FIG. 23 includes four images of ablation with a substantially linear helical wire structure electrode in cadaver tissue at differing energy and durations.

FIG. 24 contain images of a hooked helical wire structure and ablation patterns with RF energy in tissue-mimicking polyacrylamide gel phantoms during different durations.

FIG. 25 includes four images of ablation with a J-hook shaped helical wire structure electrode in cadaver tissue at differing energy and durations.

FIG. 26-A, FIG. 26-B, FIG. 26-C and FIG. 26-D are images of two linear helical wire structures coupled to the negative terminal of a DC power supply for 0, 60, 300 and 600 seconds, respectively, embedded into a tissue mimicking polyacrylamide gel phantom laced with a pH indicator.

FIG. 27 is an image of electrolysis-induced local pH change and oxygen gas evolution surrounding multiple helical wire structure anodes embedded in pH sensitive tissue-mimicking phantom, the result of applied DC voltage between anodes and saline bath return.

FIG. 28 is a fluoroscopy image of a curved helical wire structure embedded near rodent liver, with a partially insulated stainless steel interfacing needle approaching.

FIG. 29 and FIG. 30 are images of non-helical wire structure electrodes implanted in tissue. (These are identical to FIGS. 40 and 41 from PCT '374).

### FURTHER ASPECTS OF THE INVENTION

The wire structure electrodes disclosed in PCT '374 (non-helical) and PCT '007 (helical) provide tools to solve several problems with the prior methods of treating a tissue target such as a tumor or a peripheral nerve.

Prior art methods of ablation rely upon the transcutaneous insertion of probes to the tissue target so that energy conducted through the probes contacts the target directly to generate heat and destroy the tissue. Those methods are limited by the fact that probes must be relatively narrow to be inserted through the skin but their narrowness limits the size and configuration of the ablation pattern. These probes are straight (except for some which have limited also by their inability to extend “around corners” to hard to reach locations say around a blood vessel.

One aspect of the improved solution herein is to use implanted wire structure electrodes positioned at, on or in the tissue target, so that energy conducted through probes passes to the implanted electrodes which then progresses to the tissue target, where the greater resistance of the tissue creates heat for ablation. The helical and non-helical electrodes are flexible, bendable, stretchable, and the helical wire structure can take almost any shape, and as such these electrodes are configured to create larger and more complicated ablation patterns. The helical especially can create treatment patters around corners created by essential structures such as blood vessels and peripheral nerves which are not treatment targets. With these electrodes being chronically implantable, this solution also allows repeated procedures with thinner probes (i.e., needles) so that entry wounds caused by prior art probes are reduced, thereby producing less collateral damage and infection risk to healthy tissue of persons such as cancer patients who are often immunocompromised by chemotherapies and pharmaceuticals. More accurate and complete ablation can make the administration of chemotherapy more effective at lower concentrations as a result of increasing the permeability of tumor cell membranes which have been subject to ablation but not yet destroyed.

The helical and non-helical wire structure electrodes can be utilized for fiducial marking of a tumor, follow up and tracking of the exact site of tumor and treatment area. Multi-functional long term retention provides ability to treat, mark and re-treat without additional injection or skin puncture. That is, the originally injected wire structure electrode can remain in the tissue, providing the clinician the option of additional ablation follow up procedures, commonly referred to as re-ablation procedures, without need for re-insertion of a large diameter conventional probe, but instead a thin probe may be used that connects to the large surface area of the wire structure electrode. In this sense, ablation with the wire structure electrode offers: 1) repeatable procedures with thin (needle) energy conductive probe, (2) much larger surface area and (3) customizable shape. Because wire structure electrodes can integrate into the tissue and dwell for extended periods, they open several long-term treatment possibilities; re-treatment, in cases when a tumor returns after initial acute ablation, without additional injection or skin puncture; re-treatment using energy coupling; and ongoing treatment with continuous stimulation; acute stimulation in conjunction with oral, IV, intra arterial chemotherapy agents for enhanced drug effectiveness; and chronic enhancement of drug uptake of oral, IV, intra arterial chemotherapy agents. Additionally, certain classes of tumors carry a known risk of recurrence. With the current state of the art, each recurrence must be re-treated carrying similar or greater procedural and surgical risks if it is even possible to re-intervene. The wire structure electrodes address this difficulty. Because the wire structure electrodes can remain chronically in situ, they can be used as a fiducial marker for precise re-evaluation of the target site as well as repeated treatments to address recurrent tumor growth. This greatly reduces the time, trauma and cost of repeated ablative insertions and/or resection procedures.

Tumors in complicated locations may be treated by deploying a helical wire structure in a (partial or complete) half moon shape, or any other complex shape which a skillful clinician can devise. Examples of such tumors are carcinoma on the outside of—or surrounding of—an organ, a blood vessel, or in a volume that itself is more of a shape of a half moon than a round sphere.

Tissue targets include without limitation tumors of the liver, kidney, lung, and bone, as well as aberrant peripheral nerves. The present method employs electrodes comprising thin, highly conductive wires and may be loaded and deployed into or surrounding a target tissue region through a straight or nonlinear dispenser of sufficient length and diameter. The device enables tissue ablation of multiple shapes, adaptable to specific anatomy of a tumor and its surrounding vasculature or other critical structures (i.e. gallbladder, porta hepatis, bile ducts). The wire structure is capable of tissue ablation and repeated ablation through a number of energy modalities, including radiofrequency ablation (RF), direct current ablation (DC), microwave ablation (MW), laser light or high intensity focused ultrasound (HIFU).

RFA is a minimally invasive procedure used to thermally destroy tumors. Needle-like ablation electrodes are inserted into or surrounding a tumor, with electrical current (˜500 kHz) conducted between the electrode and large-surface dispersive electrodes placed on patient skin, or between electrodes in multipolar configurations. Electrical power is converted into heat by induced ionic vibrations, referred to as the joule effect. These vibrations cause cell death over an affected volume when subjected to temperatures above 60 C for several minutes. Induced high temperature leads to intracellular protein denaturation, the disruption of membrane lipid bilayers, and the coagulative necrosis of tumor cells. RFA at lower power may induce mild hyperthermia, whereby tissue is heated above the body temperature to induce physiological effects while not directly producing substantial cell death. Temperatures of 40 to 45 degrees Celsius may be maintained for times up to 1 hour, in contrast to ablative hyperthermia, which achieves temperatures greater than 55 C for shorter durations of 15 to 20 minutes. Hyperthermia treatments may result in physiological (i.e. perfusion) or cellular (i.e. gene expression) changes which improve therapeutic efficacy through localized sensitization in conjunction with a chemotherapeutic. Power may be cycled on and off over an hour-long period as a method of avoiding the transition from mild hyperthermia to ablative hyperthermia.

RF current, typically a 500 kHz alternating current, may be applied through pushing one or more partially-uninsulated thin (20-30 gauge) needles into the bulk of the wire structure electrode under image guidance. Strength of the metal-to-metal connection may be verified by an associated electrical control system, where there exists a lower impedance for the helical wire structure/ground connection compared to the partially-uninsulated needle/ground connection. Said electrical control systems prevent ablation until impedance between helical wire structure and ground, or impedance between helical wire structure and adjacent electrode, is below 1000 ohms or above 25 ohms. Needle-based current transfer may be a repeated procedure, facilitated by the secure anchoring of the wire-structure by tissue ingrowth.

A generated coagulation zone in RFA is strongly limited by heat sinks—fluid (e.g. blood) flow through vessels near a tumor causes local pockets of convective heating such that RFA is unable to achieve consistent necrosis near the tumor. For example, in the case of hepatocellular carcinoma, traditionally risky locations for treatment are tumors attached to vasculature (perivascular) which are adjacent to extrahepatic vital organs or larger intrahepatic vessels, which can considerably alter the size of the ablation zone due to the heat-sink effect, resulting in aggressive recurrences after ablation. Intravascular tumor spread along the peritumoral portal vein contributes greatly to HCC recurrence and spread. The helical wire structure is capable of surrounding vasculature closely and in a user-customized manner. Delivery of the helical wire structure may be in parallel alignment with the vasculature or hooked around the vasculature in a C or J-shape. Deploying the helical wire structure circumferentially (helically) around the vessel allows regions of focal RFA heat deposition around a perivascular tumor, essentially allowing for ablation around corners.

Coagulation zones in RFA are also strongly limited by roll-off, the cessation of RF power due to sudden increase in electrical impedance with the active electrode surrounded by desiccated tissue, which has an insulation effect. Ablation with saline infusion through cannula may limit coagulation on the electrode surface. Pulsing with RF at regular intervals, and ramping power from low wattages to mid-level power settings may help avoid charring as a result of rapid power delivery at the wire structure surface

RFA's clinical efficacy is mediated by creation of a sufficient ablative margin surrounding tumors (˜20%). Tumors may have irregular volumes that limit the use of RFA applicators, which may only produce spherical or minimally oblong ablation zones. Controlled delivery of the flexible, high geometric-surface-area (GSA) helical wire structure enables the creation of complex ablation geometries, more efficiently overlapping unconventional/non-spherical tumors. Controlled delivery of multiple helical wire structures applied around, without directly puncturing the tumor, further increases ablative volumes while avoiding unintentional scattering of tumor cells (tumor seeding.

The primary goal is to impair the target tissue: in the case of malignant tumors, permanent destruction, but in the case of peripheral nerves, only temporary impairment of neural conduction for applications such as pain relief (sensory block, afferent nerve fiber block), reduction of spasticity (motor fiber block, efferent nerve fiber block), or modulating the autonomic function of an organ, organ system or an entire individual (autonomic nerve block affecting either autonomic afferents or efferents). In the case of cancer, the goal is elimination of all viable malignant (cancerous) cells in a designated tumor volume and to provide immediate pain relief by affecting the afferent innervation into cancerous tissues. As such, ablative therapies are intended to include a 0.3 cm to 1 cm ablative margin of non-malignant tissue, in order to minimize the chance of local tumor progression or recurrence. Known risk factors of tumor recurrence post-ablation include an insufficient ablative margin, the presence of vasculature (e.g. periportal hepatocellular carcinomas (HCC)), with the odds of tumor recurrence highly correlated with larger, irregular tumors. Larger tumors, typically defined as tumors over 3 cm in diameter, oftentimes require multiple overlapping probes, applied in succession or simultaneously, to successfully achieve a sufficient ablative margin.

It is also desirable for ablative therapies to be highly precise in their effect to preserve as much normal tissue as possible. In hepatocellular carcinoma, functional hepatic reserve is a primary predictor for long-term patient survival. Well-planned ablative therapies serve to minimize damage to surrounding cirrhotic parenchyma. Preservation of nephrons in the context of renal cell carcinomas, or epithelial cells of the lung in the context of adenocarcinomas, are equally important for positive patient outcomes. The ability to preserve critical structures surrounding a tumor remains a challenge for ablation modalities.

RF, MW, laser light, or ultrasound acoustic waves are the most common sources of clinical hyperthermic ablation, generating temperatures in excess of 60 degrees Celsius. Aside from high-intensity focused ultrasound (HIFU), these energies are applied from an generated connected to needle-like applicators inserted into or surrounding the tumor. Typical lesions generated may be modeled as three-dimensional spheroids, with the major axis of the lesion aligned parallel to the applicator shaft, and two minor axes of the lesion lying perpendicular to the shaft. Though the lesion's major axis may be controlled by selecting different lengths of uninsulated applicator tips, necrosing along the minor axis is more cumbersome. Attempts to overcome this limitation have involved increasing the probe gauge, using several applicators in combination or multiple probes per applicator, and the use of expandable applicators.

As a micron-scale conductor, the wire within the helical wire structure electrode will locally produce an electric field and effective zone of heat conduction and transmission into the surrounding target tissue with target tissue fluids, and also acts as a bulk conductor, which also produces an electric field and effective zone of heat conduction. The bulk conduction (electrical/thermal) and radiation of the helical wire structure electrode therefore is the combination of both macro and micro-scale properties.

Direct current does not ablate tissue like RF which is generally associated with heat generated that kills tissue. Direct current kills not with heat but by changing the pH in the vicinity of the electrode to get cells to leak their contents as the change in acidity leading to the change in pH messes with the cell walls and the metabolism of the cells whose cell walls it does not damage right away

Electrolytic ablation/lesioning is a non-thermal technique in which a local pH change is created following application of direct current. This method has been applied towards the treatment of lung, liver, and pancreatic tumors. It has also been applied in the field of controlled nerve ablation, with nerves lesioned by DC experiencing a rapidly reduced conduction (nerve block). Applied low-voltage DC (<50V) between two or more electrodes results in electrolysis, generating hydrogen (hydronium, H₃O⁺) ions at the anode and hydroxide ions at the cathode.

Anode: 2H₂0<->O₂+4H⁺+4e⁻

Cathode: 2H₂0+2e⁻<->H₂+2OH⁻

Electrolysis also induces the movement of sodium cations towards the cathode and chloride anions towards the anode. This results in the production of sodium hydroxide and hydrogen near the cathode, and hydrochloric acid, oxygen, and chlorine near the anode. The regions surrounding the anode become acidic (pH<6), while the region surrounding the cathode becomes alkaline (pH>9), resulting in non-thermal cell death (pH<4.8, pH>10.6). Additional contributors to cell death in vivo include the generation of reactive oxygen species, though their effect is secondary to that of pH-driven cell death.

Electrolytic ablations/lesionings offer a great deal of increased precision, shaping well defined ablation margins due to the introduction of toxic levels of acid and base. Selective alteration of the local microenvironment makes it well suited as a modality for the treatment of complex tissue shapes. Helical wire structures are able to be placed precisely in user-tailored conformations, making it well suited to treat complex tumor shapes. Ease of multiple placements, such as in the potential case of a multiple helical wire structures placed as cathode returns, surrounding a single anode of tailored shape, maximizes the potential of electrolytic lesioning as a potential treatment.

The use of the helical wire structures as an embedded, indwelling implant increases the clinical relevance of electrolytic treatment by permitting the re-lesioning of complex margins without the need for multiple repeated probe insertions.

Measuring in-situ tissue electrical resistance and buffering capacity will further enhance precise lesioning. Physiologic buffering in-vivo will limit the spread of acidic and basic species following treatment completion. Electrolytic ablation may be further mediated by the flow of blood through a tissue, delivering additional buffering species and removing generated acid/base ions, further emphasizing the importance of a flexible wire structure capable of navigating around vasculature.

Electroporation, or electro-permeabilization, is the application of short pulses of strong electric fields to cells and tissues. External electric fields increase transmembrane potential, inducing the formation of nanopores, called poration. Applied voltages of up to 1 kV across electrodes introduces reversible electroporation, the formation of temporary pores in the cell membrane. Reversible electroporation has many documented applications in gene and drug delivery, where the permeabilization of the cell membrane allows the entry of molecules that would not otherwise penetrate it. Irreversible electroporation (IRE), applying voltages of up to 3 kV, results in permanent disruption of the lipid bilayer and loss of cell homeostasis. The use of small electrodes and short, repetitive electric field pulses results in a nonthermal apoptotic, as opposed to necrotic cell death, with a well-demarcated region of ablation and sharp boundaries between treated and untreated zones. IRE spares critical structures such as bile ducts, nerves, blood vessels. Pore formation does not occur significantly in tissue with higher collagenous content or elastic fiber contents. It affects only the membrane of living cells, and does not cause the denaturation or coagulation of proteins typical of thermal ablation. IRE is insensitive to the heat-sink effect. IRE generators may deliver up to 3 kV of energy in up to 100 pulses (an electric field gradient in a 40 cm³ volume of at least 800V/cm is considered the threshold for irreversible electroporation), with two or more monopolar probes or a single bipolar probe used at a time to create ellipsoid ablation zones. Multi-bipolar configurations increase the size of predictable margins. Current IRE procedures are rapid—however, they require general anesthesia and paralytics, and require synchronization of voltage pulsing with the refractory period of the cardiac cycle to avoid arrhythmias.

Creation of membrane nanopores allow permeability of agents such as chemotherapy drugs or macromolecules which would not otherwise cross the cell membrane, thus allowing for an effect upon the cell where there would otherwise be none. This allows for augmented drug/genetic delivery systems. Additionally, if enough power is transferred in a controlled field, irreversible membrane poration occurs (hence irreversible electroporation) with subsequent cell death. This is analogous to ablation, but without the thermal effects which may damage the tissue structure and scaffolding (significant vessels, ducts, critical structures). Lowering chemo load for therapeutic benefit (Irreversible/Reversible electroporation mediated increased cellular permeability) DC or rapid AC concept); Low level/chronic stimulation (external stimulator); DC or rapid short burst AC stimulation to induce damage to cancer cell membranes, allowing chemotherapy drugs to be better absorbed at potentially lower concentrations; Chemical cancer therapies require cancerous cells to uptake enough drugs to ensure their destruction. Some chemotherapeutic drugs that would otherwise be effective as a treatment may not have activity due to reduced tumor cellular uptake. Thus, drug doses that are required for tumoricidal effect may cause global damage to the surrounding healthy tissue and thus leads to unacceptable toxicity in the subject. Using specific energy delivery methods such as passing DC energy through helical wire structures causes damage to the cellular membrane of affected local cells, effectively making the cells more permeable. This technique, with well-placed electrodes, make tumor cells more susceptible to lower concentrations of cancer treatment drugs, improving effectiveness, reducing negative side effects and reducing cost.

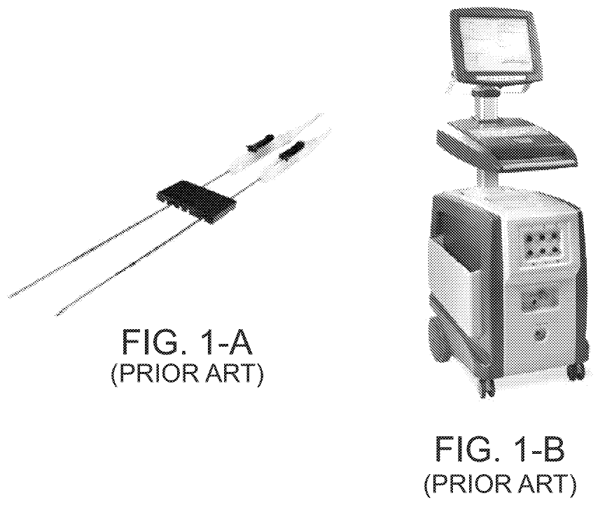

Reversible Electroporation (RE), Irreversible Electroporation (IRE) and Electrolytic lesioning (or electrolytic ablation, EA) are emerging non-thermal focal therapies. Both electroporation and electrolytic lesioning operate on the principle of an applied DC voltage. Electrolytic treatments are an area of active research in the fields of both tumor ablation and nerve blocks, with studies using bipolar-configured linear electrodes to cause chemical species evolution near the electrode surface, causing a pH-mediated localized necrosis. Prior to causing a larger volume localized necrosis, pH-mediated large volume changes (i.e. 2 to 10 mm radially away from the wire structure electrode) will first cause a neural blocking effect on afferent nerves transmitting and/or processing pain and other sensations from or through the pH-mediated localized volume as well as on efferent nerves transmitting and/or processing action/motor information to, from or through the pH-mediated localized target volume. This temporary reduction of neural activity, akin to a temporary block of neural activity, may be used as a diagnostic tool as well as a tool to determine the optimal charge delivered as direct current injected over treatment time to ensure sufficient but not over treating the target and adjacent untargeted tissues. If so desired, the direct current injection may be partially or fully reversed in either charge amount injected or in time current has been applied prior to allow for a partial temporary and a partial permanent nerve or target tissue effect as the outcome of one treatment event. Irreversible electroporation typically requires a combination of probes, with energy delivered between two probes at a time. Recorded voltages of up to 1 kV are determined reversible electroporation, inducing temporary nanopores in the cell membrane to more easily introduce genes or drugs. Recorded voltages of 1 kV through 3 kV form permanent pores which induce local apoptotic cell death. Current electroporation applicators are 19 gauge needles with 1-4 cm exposed active tips, placed parallel to one another 1-2 centimeters apart. FIG. 1-A is an image of a prior art electroporation probe and FIG. 1-B is the companion generator, Nanoknife, by AngioDynamics.

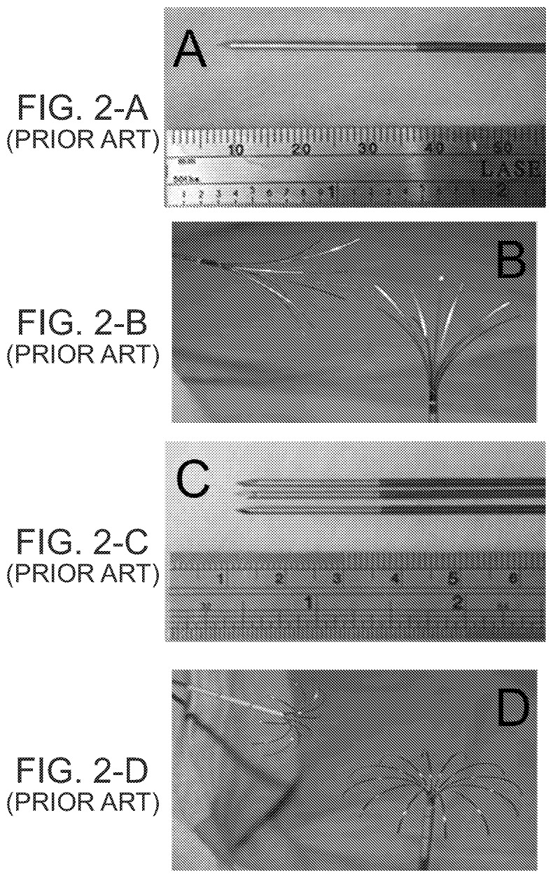

FIG. 2 contains four images of prior art devices. FIG. 2-A is an image of a 3 cm Single Active-Tip (Covidien Cool-Tip). FIG. 2-B is a StarBurst Expandable Electrode (Angiodynamics). FIG. 2-C is a Cluster Tri-Electrode 2.5 cm Active Tip (Covidien Cool-Tip). FIG. 2-D is a LeVeen Expandable Anchor Electrode (Boston Scientific).

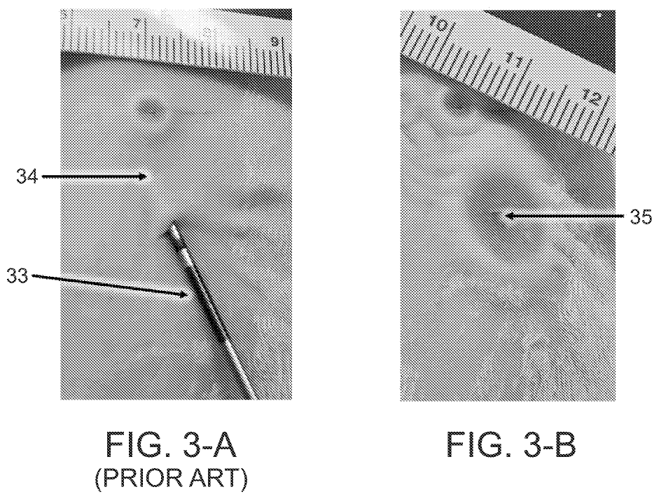

FIG. 3-A is an image of cadaver tissue with the end of a prior art RF ablation probe inserted to a subcutaneous point in the tissue to be ablated. FIG. 3-B is an image of the same cadaver after ablation and removal of the probe and showing the pattern of ablation is limited to near the end of the probe.

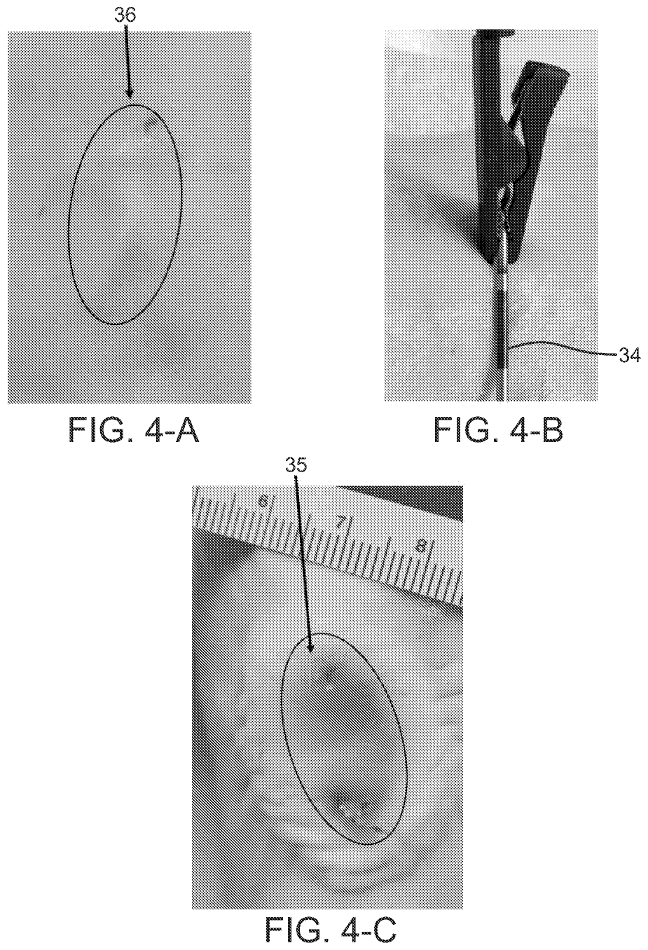

FIG. 4-A is an image of cadaver tissue with a wire structure electrode implanted subcutaneously prior to RF ablation. FIG. 4-B shows the end of a gold wire in the wire structure electrode excised and clamped directly to an RF probe. FIG. 4-C is an image of the ablation pattern in the cadaver tissue from the wire structure electrode.

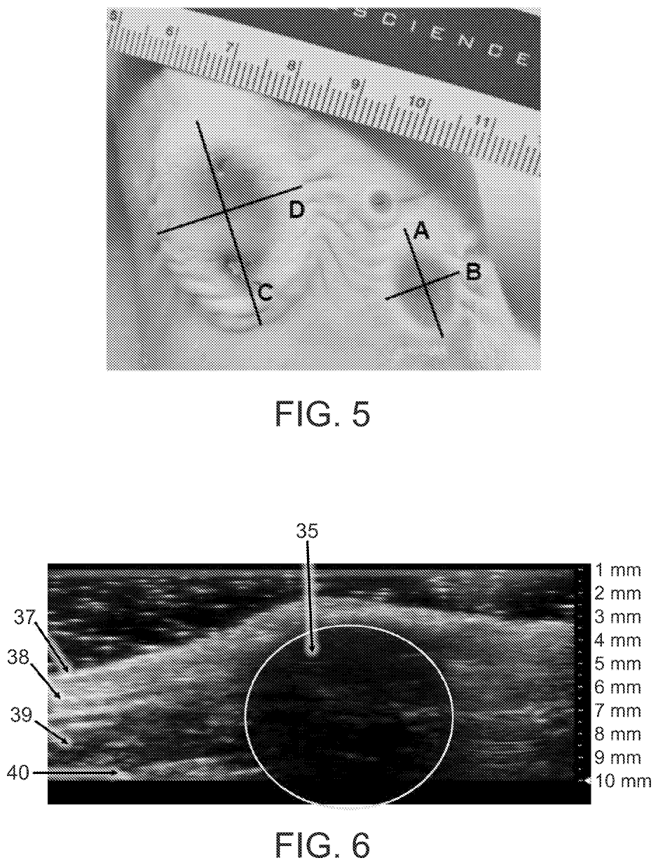

FIG. 5 is a photo showing a comparison of ablation patterns from the prior art device in FIG. 3-B and from a wire structure electrode in FIG. 4-C for the same duration and same amount of RF energy. The prior art device pattern is shown by axes A (16.7 mm) and B (10.6 mm) with an approximate total area of 139 mm², and the present invention's pattern is shown by axes C (33.6 mm) and B (23.0 mm) with an approximate total are of 607 mm².

FIG. 6 is an ultrasound visualization of a pattern of RF ablation with the present invention for 20 watts and 120 seconds.

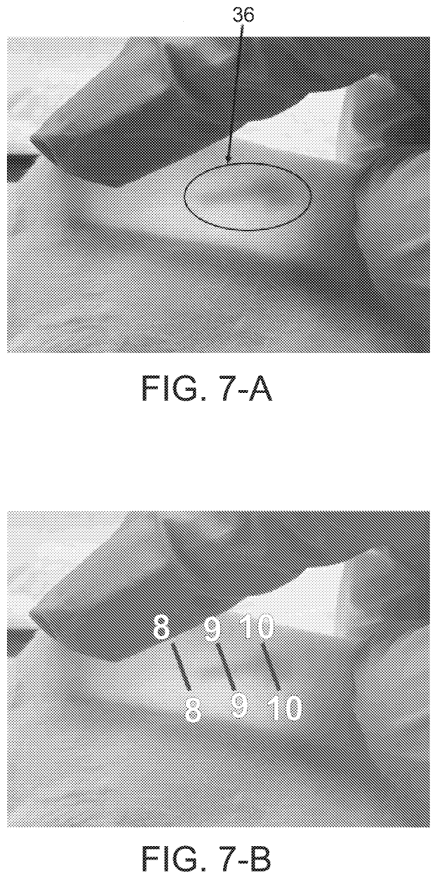

FIG. 7-A is transdermal imaging of a subcutaneously implanted gold wire structure electrode in a J-hook shape, and FIG. 7-B is the same image with cross sections 8-8, 9-9 and 10-10 labeled.

FIG. 8 is an ultrasound image of the cross section 8-8 in FIG. 7-B showing none of the wire structure electrode, taken with a VEVO 3100 system and 45 MHz probe.

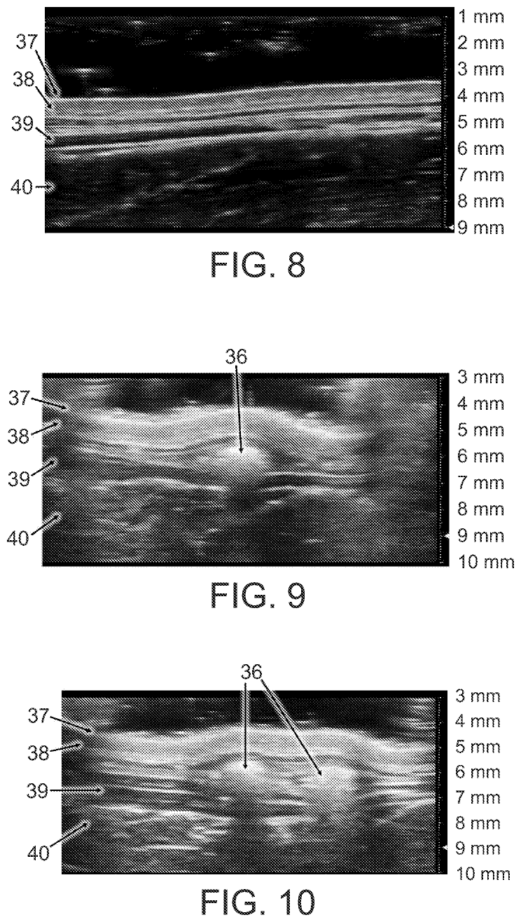

FIG. 9 is an ultrasound image of the cross section 9-9 in FIG. 7-B showing only the long shaft of the J, taken with a VEVO 3100 system and 45 MHz probe.

FIG. X is an ultrasound image of the cross section X-X in FIG. 7-B showing the long shaft and hook of the J, taken with a VEVO 3100 system and 45 MHz probe.

The present invention uses, in one embodiment, a flexible multi-stranded helical wire structure of materials which display desirable thermal conductivity, electrical conductivity, and heat capacity, making the device suitable for efficient electrical coupling, heat transfer, or electric field distribution. The manufacturing of a helical wire structure electrode outside the body may involve steps of rolling and/or folding. Individual strand diameters in the wire structure preferentially range from 25-75 microns. Incorporating strands of greater thicknesses is a method of mechanical optimization for increasing rigidity of the helical coil, or creating permanent curvatures at certain points along the length of the wire structure upon deployment.

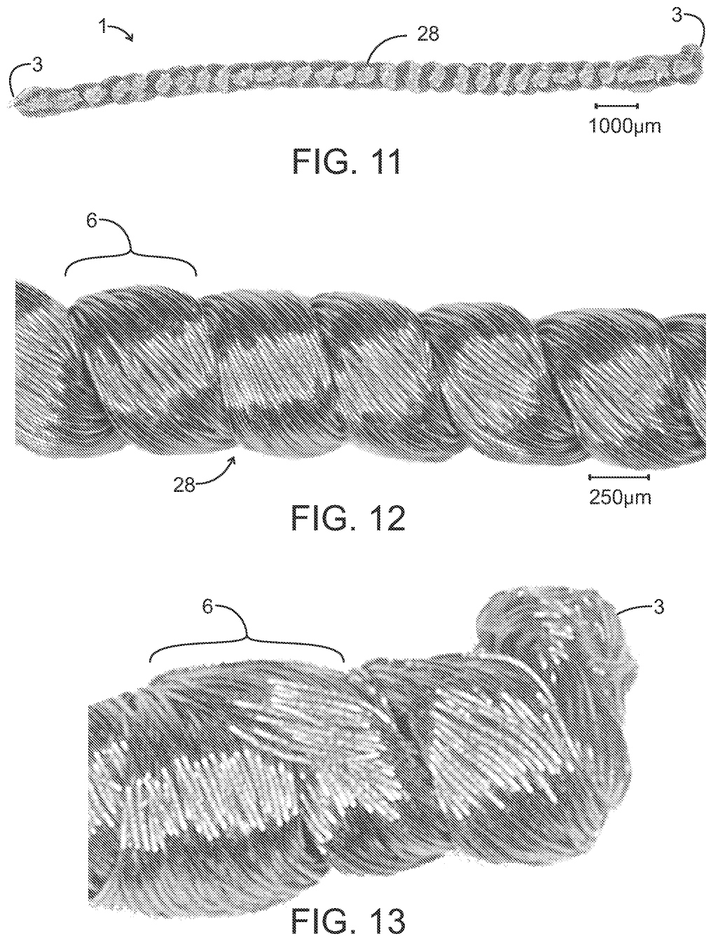

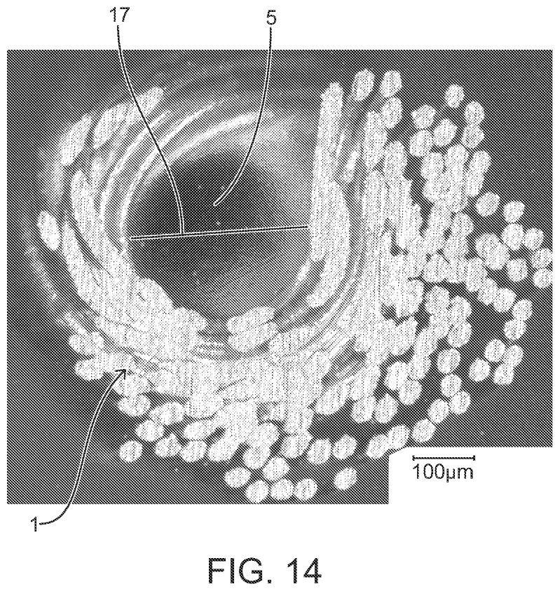

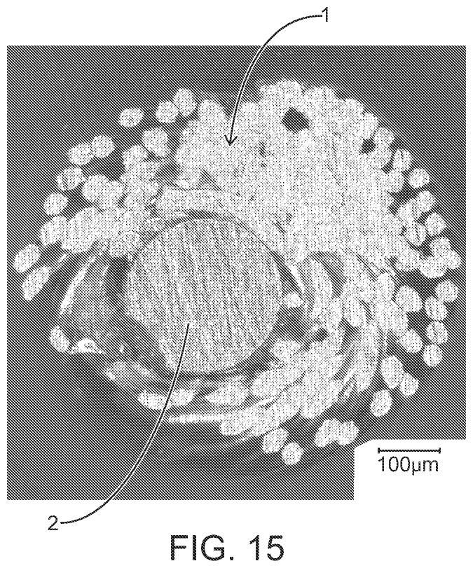

FIG. 11 is an image (15.6×) of one embodiment of the injectable electrode with the helical wire structure after removal of the guidewire. The wire rope is 100 strands of 25 diameter micron gold wire and the helical wire structure has an approximate outer diameter of 0.75 mm. Overall length is approximately 2 cm and is made from 6 meters of continuous gold wire. FIG. 12 is a closer image (100×) of a middle portion of the helical wire structure of FIG. 11. FIG. 13 is a closer image (100×) of a rounded end of the helical wire structure of FIG. Y. FIG. 14 is an image (300×) of a latitudinal cross-section of an electrode comprising a helical wire structure comprising a wire rope comprising 100 strands of 25 micron diameter gold wire. FIG. 15 is the same electrode as in FIG. 14 before the 0.25 mm guidewire was removed.

The helical wire structure, loaded into a needle (linear or curved passive introducer) or flexible catheter (steerable, active introducer), is capable of deployment via the use of a bendable plunger, sufficiently dense hydrogel, or similar methods of pushing the coil within the needle such that the coil exists at a constant, predictable rate. Insertion of the needle or flexible catheter may create a void in soft tissue by displacement. Bodily fluid (e.g. blood) ingress into the void is a natural consequence of the insertion of an electrode, and may be accompanied by the introduction of saline or a hydrogel, or gaseous microbubbles in the context of contrast-enhanced ultrasound. The helical wire structure inside the needle or flexible catheter may be combined with liquid, gel, gas, or a mixture of the three, to pass around or through crevices between the helix and the wall of the introducer, or through the centerline of the helix itself. Hypertonic saline injection in the context of ablation increases ionicity and conduction within the tumor, thereby increasing ablative volumes while preventing tissue desiccation that would otherwise limit ablation volume.

One or more helical wire electrodes may be injected centrally within the tumor site, or at multiple oblique sites tangential/adjacent to the targeted tumor to create different polar arrangements for selective heating or application of an electric field across wire electrodes. Once placed, the helical wire structure is intended to reside within the tissue. High contrast/radiopacity exhibited by the helical wire structure enables accurate localization of the tumor and previous treatment area under computed tomography or fluoroscopy. Re-interfacing and retreatment through the helical wire structure can be accomplished through electrical coupling with an external energy source, and subsequent hyperthermal or non-thermal modes of ablation.

Both hyperthermic and non-thermal ablation modes require high spatial targeting accuracy. Predictability is crucial in balancing the critical need to apply irreversible damage to a whole tumor with reduction of harm to surrounding critical structures. A variety of models exist which seek to mimic properties of biological tissue as simple, highly reproducible methods of evaluating device performance and creating therapy protocols, thereby eliminating the need for animal and human subjects. Ex vivo tissues are the most common method of assessing a treatment volume. However, the heterogeneous nature of ex-vivo tissue makes reproducible characterization difficult. Assessments of ablation therapies using ex-vivo tissues require cutting, staining, and subjective observation of tissue. Other efforts have focused on the creation of tissue-mimicking phantoms for quantitative and predictive measures of device performance. In evaluating hyperthermic ablation modes, proposed formulations have used agarose and polyacrylamide gels incorporated with heat-sensitive materials such as bovine serum albumin and thermochromic compounds (liquid crystals, leuco dyes, or permanent color change inks). Gels such as agarose and polyacrylamide (PAG) have desirable properties, including melting points higher than those achieved through ablation, and the ability to be doped with materials to mimic properties such as electrical and thermal conductivity. The use of polyacrylamide gels altered with sodium chloride and permanent color changing dyes allows for visualization and quantitative assessment of heat-affected zones caused by an electrode. The use of polyacrylamide gels altered with pH indicators (i.e. phenol red) allows for visualization and quantitative assessment of locoregional pH changes surrounding an electrode. Temperatures as well as acidity may be backtraced through post-experimental image analyses and verified through the placement of thermocouples, fiber-optic thermometers, or micro-pH electrodes. This allows for the creation of models that predict temperature or pH change in response to supplied power for specific amounts of time, aiding in therapeutic planning for both thermal (RFA, MW, Laser, HIFU) and non-thermal (IRE, EA) modes of ablation.

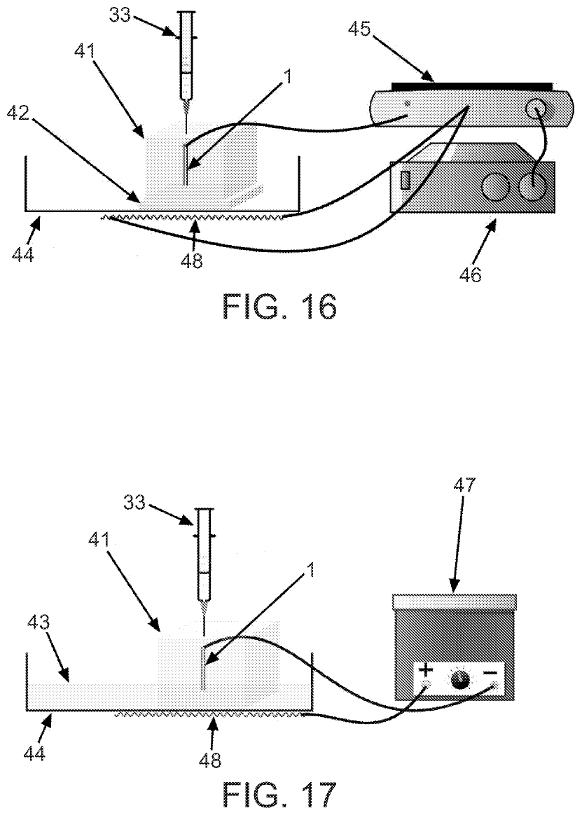

FIG. 16 is an RF ablation experimental set-up. The helical wire structure electrode is either injected via dispenser or may be pre-embedded into a temperature sensitive gel phantom. Connection between RF generator and helical wire structure may be made via direct contact using a 30 g needle.

FIG. 17 is a DC ablation experimental set-up. The helical wire structure electrode is either injected via a dispenser or may be pre-embedded in a pH sensitive gel phantom. The negative terminal of the DC supply is attached to a helical wire structure electrode via direct contact (30 g needle). The positive terminal is attached to the saline-filled grounding plate for return.

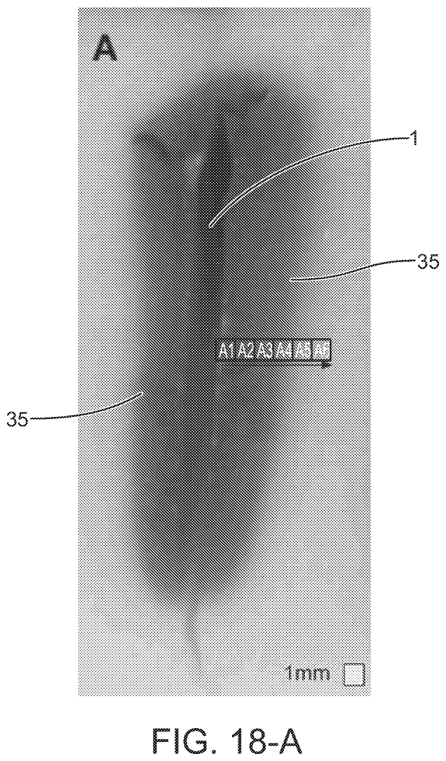

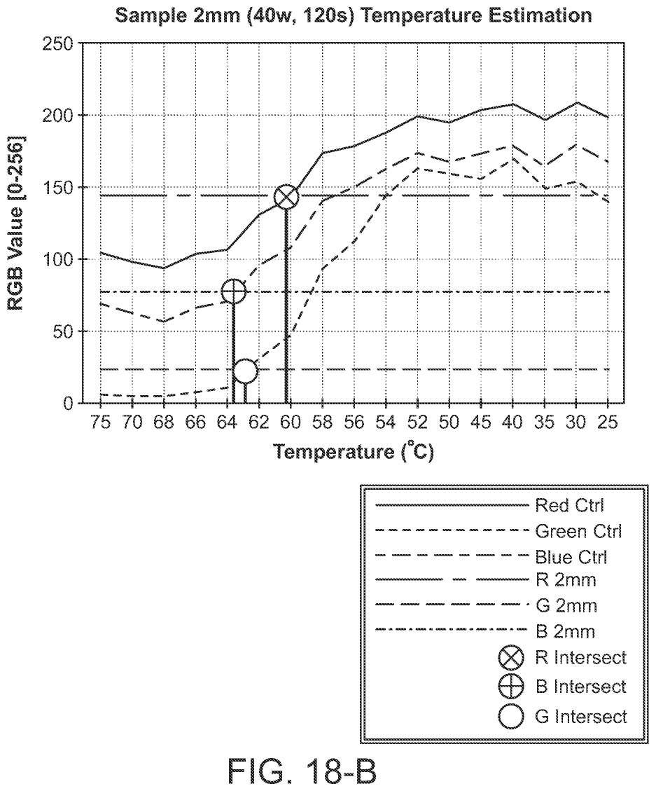

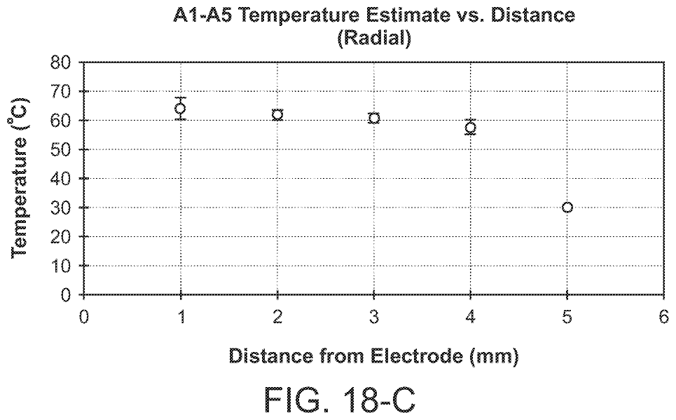

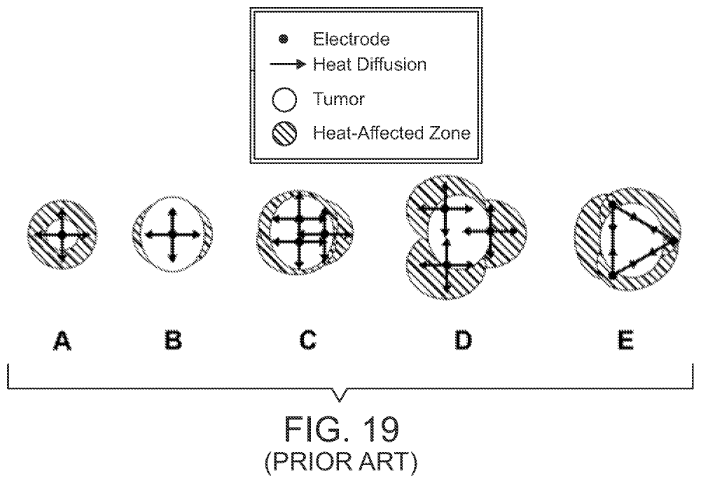

FIG. 18 addresses post-experiment image-based temperature estimation in application of RF energy. FIG. 18-A is an image of an ablative zone effected by a 2 cm-length helical wire structure electrode subjected to 40 W power over two minutes. FIG. 18-B shows temperature estimation based on RGB value found 2 mm from electrode site compared to RGB value of control set across 25-70 degrees Celsius. FIG. 18-C shows temperature estimation vs. distance at sites 1-5 mm from the center of the ablative zone.

FIG. 19 shows prior art electrode placement and heat dispersion— bird's eye view. FIG. 19-A shows linear electrode placement, radial heat dispersion for a small (<3 cm) tumor, and complete ablation. FIG. 19-B shows linear electrode placement, radial heat dispersion for a large (>3 cm) tumor, but incomplete ablation. FIG. 19-C shows multiple probe placement (intratumoral) required to cover larger spheroid tumor. FIG. 19-D shows alternative multi-probe arrangement, inconsistent tumor margin for intratumoral placement. FIG. 19-E shows “no touch” ablation, probes arranged outside the tumoral space, with current/heat diffusion running between each electrode to achieve consistent tumor margin.

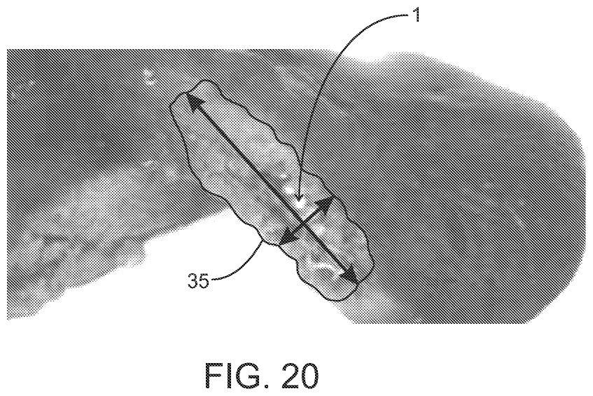

FIG. 20 is an image of rat liver left lobe subject to 1 cm helical wire structure electrode placement and RFA at 20 W over 1 minute. The arrows show the extent of the ablation: width of ˜3 mm, and length of ˜15 mm.

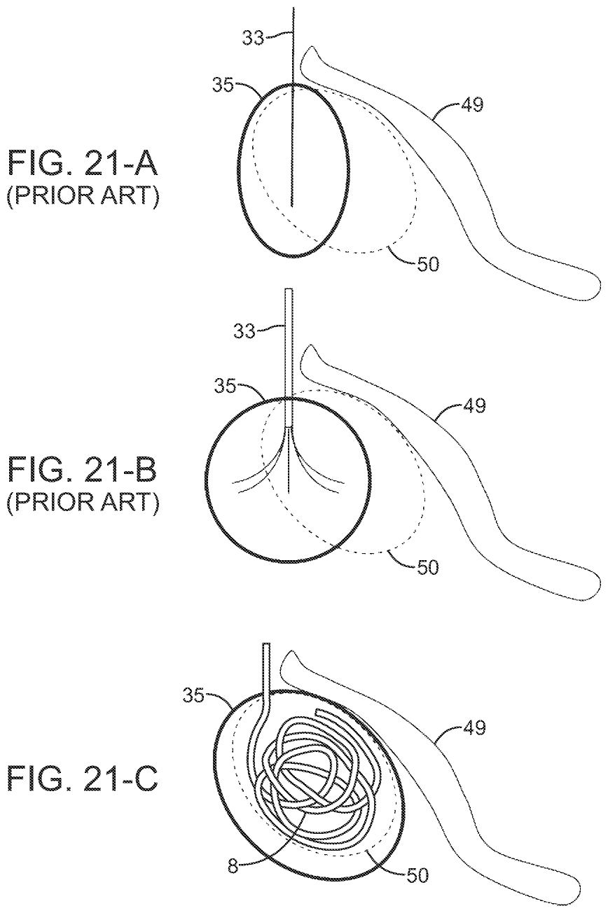

FIG. 21 contains three schematics for “around a corner” procedures for a tumor (in dotted lines) shielded by a blood vessel comparing prior art ablation devices and outcomes with the present invention and outcome. FIG. 21-A is a schematic of a prior art single probe and its pattern (oval with solid line) which affects only part of the tumor. FIG. 21-B is a schematic of a prior art single probe with a starburst and its pattern (circle with solid line) which affects only part of the tumor. FIG. 21-C is a schematic of a helical wire structure electrode and its pattern (tilted larger oval with solid line) which affects all of the tumor. By introducing the helical wire structure into a tumor in the shape of the tumor and close to the center line of the tumor (while staying far enough away from vital structure such as the blood vessel that is not to be damaged by the application of RF ablation for example), a treatment may be provided to a patient that would otherwise not be possible to treat the tumor and leave the blood vessel intact. Similar scenarios are a complex shaped tumor in difficult regions to access with a “straight” ablation probe.

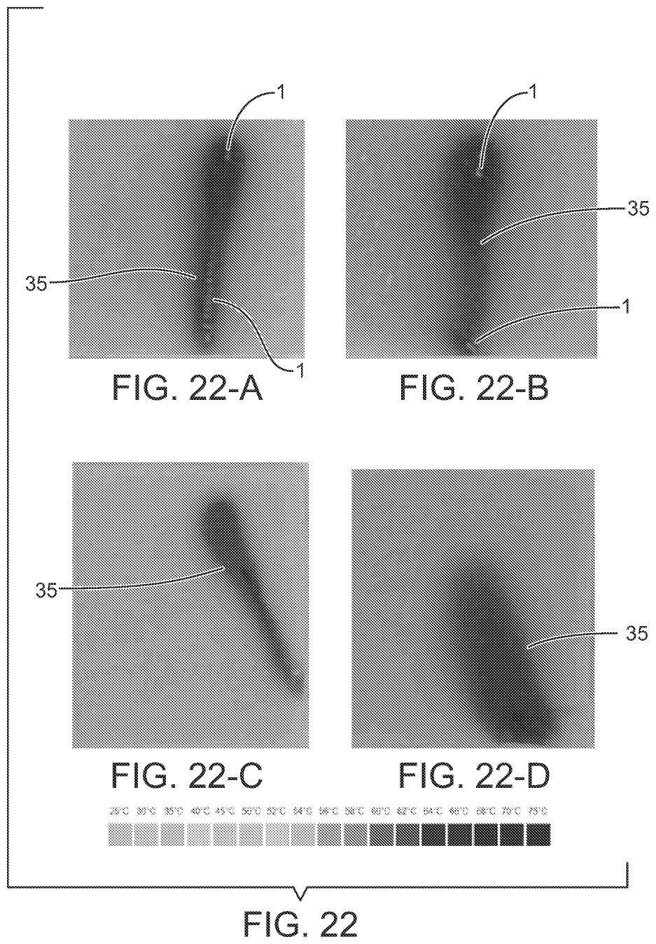

FIG. 22 contains four images of patterns of radiofrequency ablation for linear helical wire structures in tissue-mimicking polyacrylamide gel phantoms laced with a thermochromic ink, along with a temperature scale. FIG. 22-A received 10 watts for 60 seconds, FIG. 22-B received 10 watts for 120 seconds, FIG. 22-C received 20 watts for 60 seconds, and FIG. 22-D received 20 watts for 120 seconds.

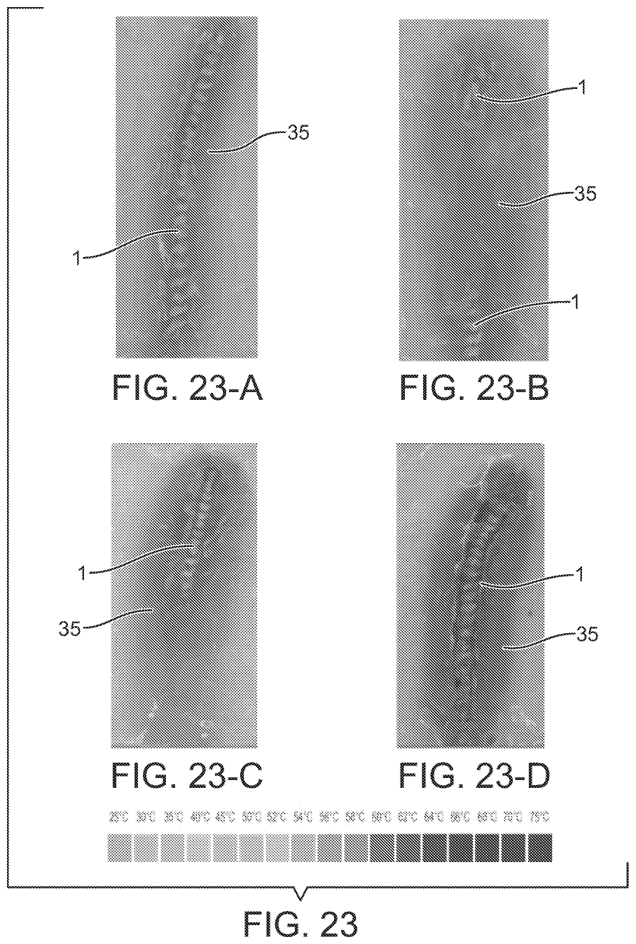

FIG. 23 includes four images of ablation with a substantially linear helical wire structure electrode in cadaver tissue as follows: FIG. 23-A, 20 watts for 60 seconds; FIG. 23-B, 20 watts for 120 seconds; FIG. 23-C, 40 watts for 60 seconds; and FIG. 23-D, 40 watts for 120 seconds.

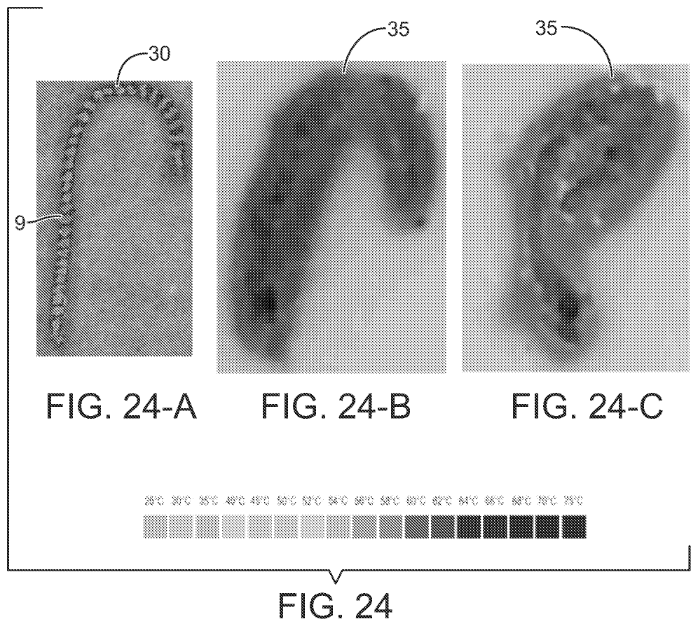

FIG. 24 contains images of curved ablation patterns with RF energy in tissue-mimicking polyacrylamide gel phantoms. FIG. 24-A is a photo of a helical wire structure in “hook” conformation prior to implantation. FIG. 24-B shows the implanted helical wire structure of FIG. 24-A and the affected pattern after being subjected to 40 watts of over 60 seconds. FIG. 24-C shows the same implanted helical wire structure subjected to 40 watts over 120 seconds, showing a larger affected pattern than in FIG. 24-B. The focal ablative region expands from the center of curvature of the implanted helical wire structure.

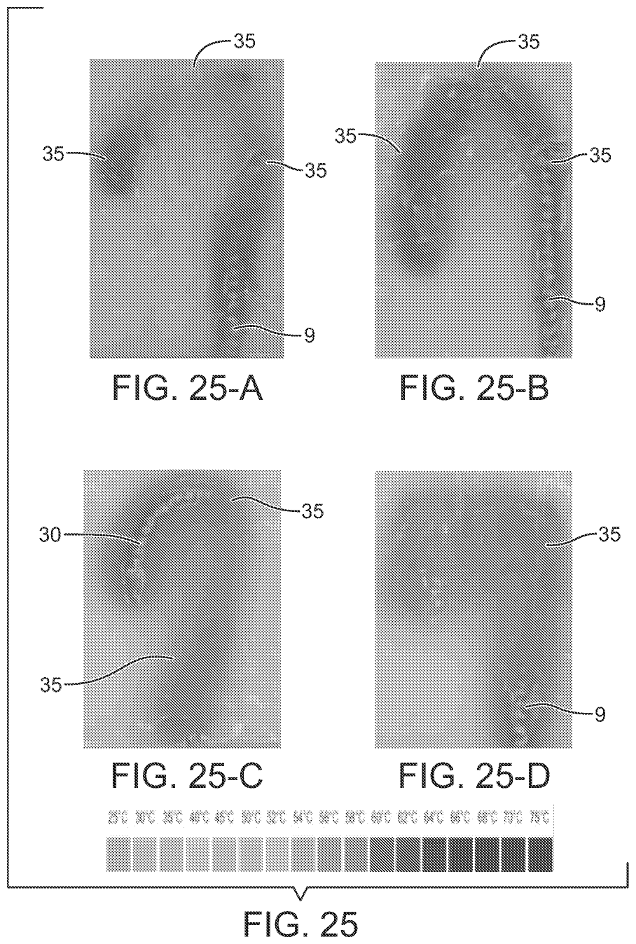

FIG. 25 includes four images of ablation with a J-hook shaped helical wire structure electrode in cadaver tissue as follows: FIG. 25-A, 20 watts for 60 seconds; FIG. 25-B, 20 watts for 120 seconds; FIG. 25-C, 40 watts for 60 seconds; and FIG. 25-D, 40 watts for 120 seconds.

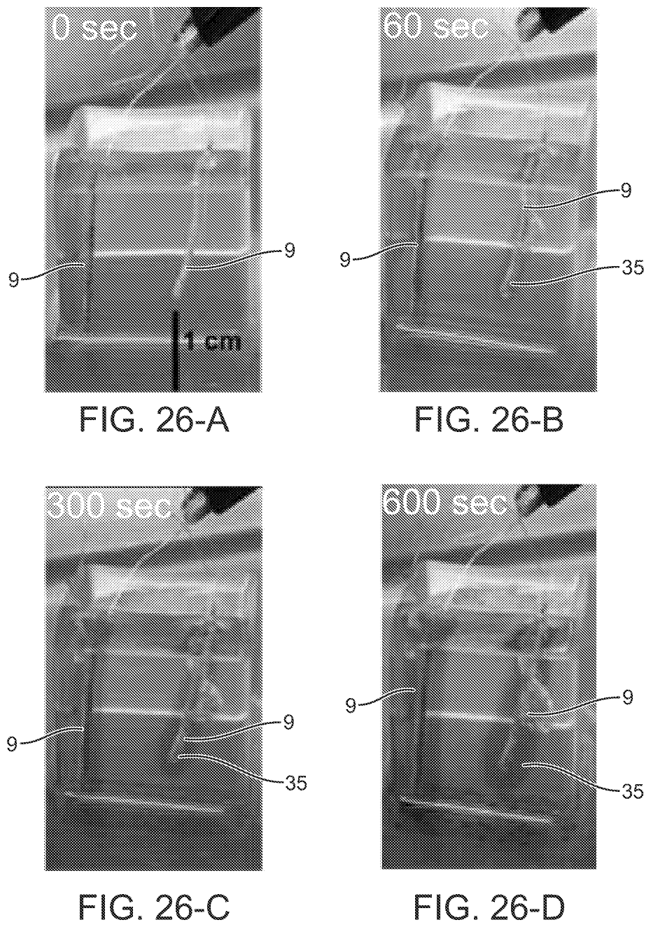

FIG. 26-A, FIG. 26-B, FIG. 26-C and FIG. 26-D are images of two linear helical wire structures coupled to the negative terminal of a DC power supply for 0, 60, 300 and 600 seconds, respectively, embedded into a tissue mimicking polyacrylamide gel phantom laced with a pH indicator. The gel phantom is in a container surrounded by saline, to which the positive terminal of the power supply is connected such that the saline acts as the return. 10V DC is supplied over the course of 600 seconds and a pH decrease due to the production of acidic species (H+, HCl in particular) is observed across both helical wire structures emanating radially, alongside oxygen gas evolution (bubbles). Through use of a battery or a DC power supply set to a constant potential, the negative terminal may be connected via needle or partially-insulated clip to anode (helical wire structure). The cathode (positive terminal) may be connected to an adjacent site, either a conductive bath or an tangentially placed electrode.

In ablation, lidocaine is often injected at the ablation location prior to applying ablation. With the implanted helical wire structure electrode which is implanted prior to the ablation procedure, instead of lidocaine injection the clinician may pre-treat with slowly ramped DC to block the sensory innervation of the tissue prior to DC Ablation or RF ablation.

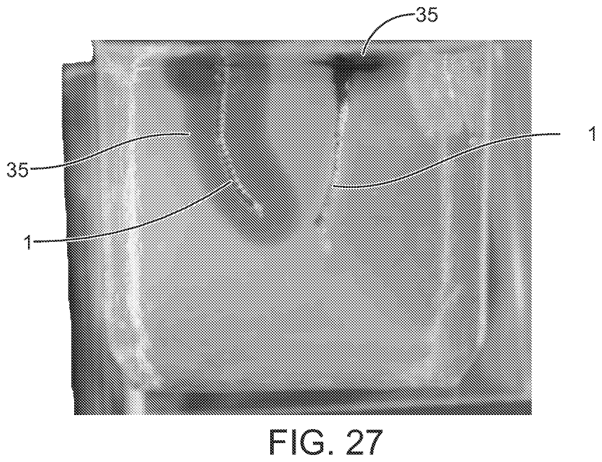

FIG. 27 is an image of electrolysis-induced local pH change and oxygen gas evolution surrounding multiple helical wire structure anodes embedded in pH sensitive tissue-mimicking phantom, the result of applied DC voltage between anodes and saline bath return. Helical wire structure, electrolysis-induced local pH decreases at anode (left) and pH increases at cathode (right), accompanied by surrounding oxygen and hydrogen gas evolution at anode and cathode, respectively, are the result of applied DC between anode and cathode return. The structure of the anode emphasizes that the helical wire structure is capable of non-linear pH change geometries.

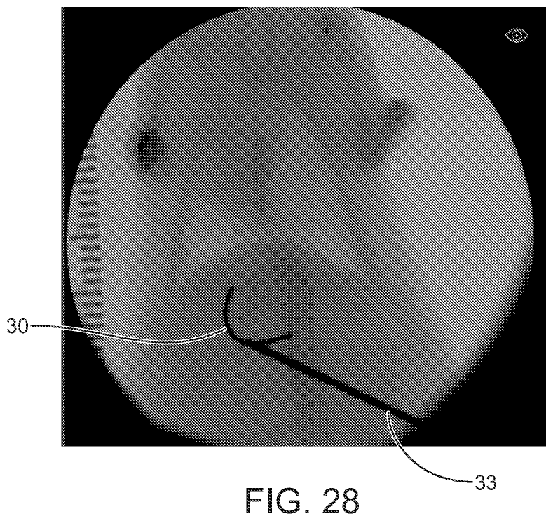

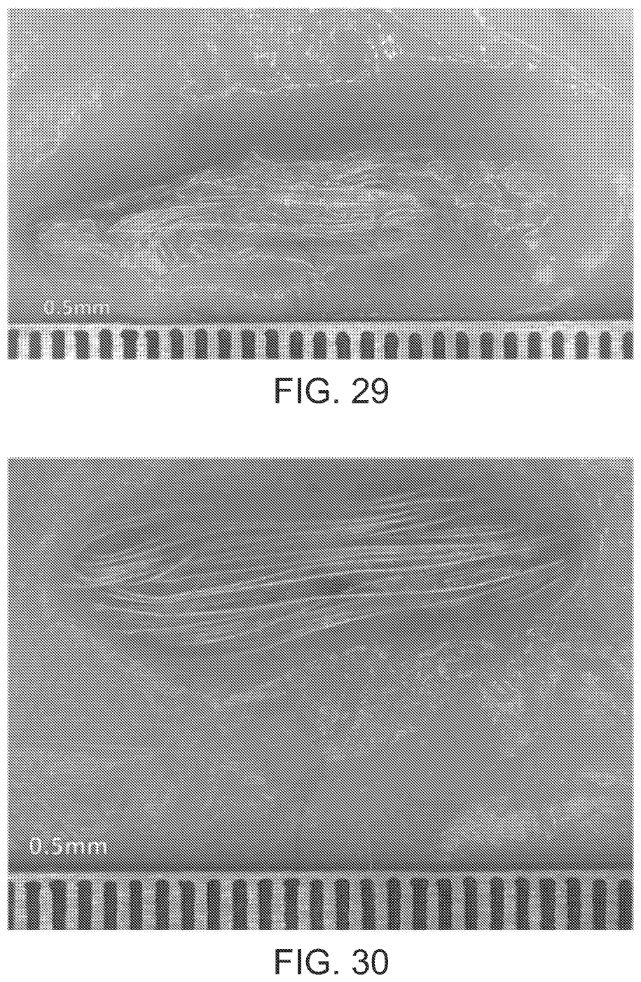

FIG. 28 is a fluoroscopy image of a curved helical wire structure embedded near rodent liver, with a partially insulated stainless steel interfacing needle approaching.

Amplitude and gradient of a generated field depends on the applied voltage and the distance between the electrodes. The tailored delivery of the highly conductive helical wire structure allows the user to introduce electrodes in a close arrangement to either irreversibly or temporarily electroporate tissue. Helical wire structures are also not restricted to fixed geometries, again, making it simple to ensure that the target is entirely enclosed within an applied field. Being an indwelling device with a predictable field output allows repeated interfacing and sensitization of tissue through reversible electroporation to a combination with chemical therapeutics, prior to a hyperthermic or non-thermal ablative treatment. Sensitization using electroporation through the helical wire structure decreases the required load of drug, reducing side effects and cost.

Energy may be applied to the helical wire structure through a specially designed high voltage generator and a secure, well insulated needle interface, as described. Design of power systems for electroporation devices requires a great deal of attention to safety due to the high energies accumulated in capacitors and from the delivery of high electrical currents to the patient—both operator and patient are at some risk of electrocution if energy release to the patient is not reliably controlled. The resistive load of a biological tissue varies, and depends on the physical properties of the electrodes. In the case of the helical wire structure, this may be difficult to assess without a preliminary test. Existing electroporation devices which measure a load of more than 50 A will interrupt the pulse sequence, under the assumption that a short circuit or sparking is occurring between electrodes.

MW ablation is a thermal technique which creates an electromagnetic field surrounding a monopolar electrode, inducing homogeneous heating and coagulative necrosis. It heats rapidly, reaching higher temperatures than other hyperthermia methods (RF), and can treat larger ablation areas compared to monopolar RF. It achieves higher temperatures faster compared to RF, and is less sensitive to the heat-sink effect. However, it is difficult to define a reliable end-point to set the amount of energy deposition during MW. Multiple linear applicators increase the risk of injuries and complications resulting from over-ablating. Helical wire structure, indwelling, serves as a fiducial marker for clinicians to easily locate and re-evaluate the target site for required repeat treatments.

HIFU is used to cauterize tissue using 5 W/cm² or greater power. HIFU has difficulty as a standalone therapy, due to poor rates of complete ablation, resulting in higher rates of recurrence. A helical wire coil as a chronically indwelling implant allows easy relocation and accurate targeting for repeat treatments. HIFU introduces biological effects upon tissue (most commonly thermal effects and ablation). Typically this occurs with specific energy deposition of 5 W/cm² and greater. Additional biological effects within the sub thermal envelope may enhance drug/gene delivery mechanisms similar to electroporation as discussed herein. Currently HIFU as an ablation technique is not complete enough to be completely curative in treating malignant tumors. HIFU has a good use case for benign tumors such as fibroid tumors of the uterus, because it can be useful to mechanically shrink down the bulk of the tumor. However, while HIFU can shrink a tumor through partial tissue destruction, studies of effectiveness indicate it is unlikely to achieve complete ablation. The failure mode of ablation as a treatment for malignant tumors can be due to microscopic disease that we can't image well enough to see. Non-fully ablated malignant tumors almost always regenerate and regrow. One embodiment of the present invention resonates external ultrasound off the solid focal point of the wire structure electrode to create thermal energy and thus strengthen the ablative ability of the ultrasound signal. Fiducial marking and visibility provides for accurate tracking for recurrence and retreatment at the target site.

The present invention in other embodiment includes temporary placement of the helical or non-helical wire structure electrodes.

### FURTHER ASPECTS OF THE INVENTION

In addition to the foregoing, the invention also includes additional aspects as follows.

A method of treating a tissue target in a body comprising the steps of implanting a wire structure electrode in, on or near the tissue target, said wire structure electrode being biocompatible and capable of chronic implantation in said body, providing grounding means exterior to said body, contacting the wire structure electrode through a percutaneous probe, said probe being energy conductive, and delivering energy through said probe to said tissue target, said energy being sufficient to lesion or destroy said tissue target, and withdrawing the probe from the body. In another embodiment the method further comprises at the end a step of imaging the wire structure electrode as implanted as a fiducial marker and then repeating all steps after implanting the wire structure electrode. When the tissue target is a peripheral nerve, one embodiment of the method is a new step after imaging of gradually ramping up direct current to the peripheral nerve from zero mA to 0.1 mA in less than 30 seconds and thereafter increasing as needed to prevent pain. Using the same method, the wire structure electrode comprises a helical wire structure or a non-helical wire structure, the latter being selected from the group consisting of rolled, folded, extruded and the like. The energy delivered through the probe may be selected from the group of radiofrequency current, microwave energy, direct current and changing magnetic field inducing alternating currents, and is sufficient to heat said tissue target to at least 60 degrees C. In another embodiment said energy delivered through the probe is direct current and is sufficient to lesion said target tissue through an induced pH 4 or less or 10 or more. In yet another embodiment of the method, energy delivered to the probe is current and produces reversible electroporation, irreversible electroporation or electrolytic lesioning.

In yet another aspect, the amplitude of the direct current to achieve the lasting pH change is reached by initially slowly ramping the DC from zero mA to 0.1 mA in less than 10 seconds, thereafter increasing the slow rate as applicable such that the patient does not report sensation of DC applications to avoid the need for pharmacological anesthetic agents consisting of lidocaine, marcaine, or similar as the means of reducing pain during the application of DC to block pain perception while applying treatment to the tissue.

The invention also embodies a system to treat a neurological condition comprising an implantable folded wire structure intended to be left in place chronically that can fold up or bunch up or fold in when brought in mechanical contact with the biological target tissue or surrounding tissues, an percutaneous interfacing device intended for the temporary penetration of skin to conduct energy from outside of the body to the implanted folded wire structure inside the body, the interfacing device having a skin penetrating insulated portion and an uninsulated portion, both portions intended to penetrate the skin, the insulated portion penetrating the outer layers of the skin and the uninsulated portion intended to interface with the chronically placed folded wire structure, a connector to an energy signal generation device, and a signal generation device providing the energy, and the signal generation device providing the energy being a radiofrequency generator, a microwave generator, a direct current generator. In another embodiment of the system, the signal generation device providing the energy being a direct current and a radiofrequency generator, the device being capable to first generate a ramped direct current to sensory block neural tissue in the vicinity of the implanted folded wire structure electrode, and second being able to provide a radiofrequency energy to ablate target tissue in the vicinity of the implanted folded wire structure electrode.

The invention also is a method of treating cancerous tissue by applying an electrical energy signal to cancerous tissue with the aid of a chronically implanted folded wire structure in close timed proximity to the application of chemotherapy agents to a cancer patient, the treatment consisting of multiple applications of either treating cancerous tissue with RF, MW, or DC energy alone, or treating cancerous tissue with RF, MW, or DC energy in close time proximity with therapeutic agents such as chemotherapeutic drugs.

Another method is included within the invention wherein the implanted folded wire structure enables easy repeat treatment via RF, MW or DC energy that is being delivered by a 24 gauge or smaller needle, allowing for a patient-friendly repeat treatment.

We claim:

1. A method of treating a tissue target in a body comprising the steps of
a. implanting a wire structure electrode in, on or near the tissue target, said wire structure electrode being biocompatible and capable of chronic implantation in said body,
b. providing grounding means exterior to said body,
c. contacting the wire structure electrode through a percutaneous probe, said probe being energy conductive, and
d. delivering energy through said probe to said tissue target, said energy being sufficient to lesion or destroy said tissue target, and
e. withdrawing the probe from the body.

2. The method of claim 1 further comprising step f of imaging the wire structure electrode as implanted as a fiducial marker and then repeating steps b-e.

3. The method of claim 2 wherein the tissue target is a peripheral nerve, further comprising a new step after step f and before repeating steps b-e comprising gradually ramping up direct current to the peripheral nerve from zero mA to 0.1 mA in less than 30 seconds and thereafter increasing as needed to prevent pain.

4. The method as in claim 1 wherein the wire structure electrode comprises a helical wire structure.

5. The method as in claim 1 wherein the wire structure electrode comprises a non-helical wire structure.

6. The method as in claim 4 wherein the non-helical wire structure is selected from the group consisting of rolled, folded, extruded and the like.

7. The method as in claim 1 wherein said energy in step d is selected from the group of radiofrequency current, microwave energy, direct current, high intensity focused ultrasound and changing magnetic field inducing alternating currents, and is sufficient to heat said tissue target to at least 60 degrees C.

8. The method of claim 1 wherein said energy in step d is direct current and is sufficient to lesion said target tissue through an induced pH 4 or less or 10 or more.

9. The method of claim 1 wherein said energy in step d is current and produces reversible electroporation, irreversible electroporation or electrolytic lesioning.
